(19) United States
(12) Patent Application Publication
(10) Pub. No.: US 2026/0137870 A1
(43) Pub. Date: May 21, 2026

(54) THERANOSTIC DOSE ADMINISTRATION CADDY

(71) Applicant: The Research Foundation for the State University of New York, Albany, NY (US)

(72) Inventors: David Lubin, Syracuse, NY (US); David Feiglin, Syracuse, NY (US); Kurtis Wilson, Syracuse, NY (US); Leen Alkukhun, Syracuse, NY (US); Garrett Depalma, Syracuse, NY (US); Kent Ogden, Cazenovia, NY (US); Mary McGrath, Syracuse, NY (US); Paul Depew, Syracuse, NY (US); Zohaib Khan, Syracuse, NY (US); Meghan Stanton, Syracuse, NY (US)

(21) Appl. No.: 19/394,230

(22) Filed: Nov. 19, 2025

Related U.S. Application Data
(60) Provisional application No. 63/722,198, filed on Nov. 19, 2024.

(51) Int. Cl. A61M 5/178; A61N 5/10; G21F 5/018

(52) U.S. Cl. CPC A61M 5/1785; A61N 5/1001; G21F 5/018; A61N 2005/1021; A61N 2005/1094

(57) ABSTRACT

A theranostic dosage caddy is disclosed. The dosage caddy includes a body having a proximal end, a distal end, a working surface, and a supporting surface configured to rest upon a stable surface. The dosage caddy also includes a compartment formed in the working surface of the body and configured to hold a syringe, as well as a distal protrusion at the distal end of the body adjacent to the compartment, where the distal protrusion is configured to hold a distal end of the syringe. The proximal end of the body includes a cavity configured to pass an extender rod therethrough to selectively engage a plunger of the syringe. Additionally, the dosage caddy includes a retention lip on the supporting surface of the proximal end of the body. The retention lip is configured to prevent the supporting surface of the body from sliding upon the stable surface.

### CROSS-REFERENCE TO RELATED APPLICATION

This invention claims the benefit of U.S. Provisional Patent Application No. 63/722,198, filed on Nov. 19, 2024, the entirety of which is hereby incorporated herein by this reference.

### BACKGROUND

The present disclosure generally relates to medical equipment, and more particularly, to a theranostic dosage caddy particularly configured to safely administer radioactive therapeutic agents.

Radioligand theranostics comprise a cutting-edge field of nuclear medicine which combines therapeutic α and β radiation with a specific targeting molecule. The recent advances in peptide receptor radionuclide therapy (PRRT) have revolutionized the field of targeted cancer therapy. With the widespread introduction of 177Lutetium DOTATATE (Lutathera) for treatment of gastrointestinal neuroendocrine tumors and of 177Lutetium Vipivotide Tetraxetan (Pluvicto) for treatment of prostate cancer, clinical facilities have been transformed into radioligand therapy centers.

Currently, the FDA has approved two such radioligand therapies for use in cancer treatment, Lutathera (177Lutetium DOTATATE) and Pluvicto (177Lutetium Vipivotide Tetraxetan). One of these therapies, Pluvicto, is currently dispensed from the radiopharmaceutical distributor in a 20 cc syringe for intravenous injection administration. Currently, there is no reasonable way to allow for reliable administration of the dose without exposing the healthcare professional to radiation. The healthcare professional holds the syringe in a syringe shield during the 2-minute intravenous injection, and even with lead-lined gloves, this set up gives significant radiation exposure to the treating physician (Table 1). Further, the lead-lined gloves limit dexterity such that the syringe shield is not secure when grasped with the lead-lined gloves, thus trading security and control for radiation exposure.

### BRIEF DESCRIPTION

A first aspect of the disclosure provides dosage caddy, including: a body having a proximal end, a distal end, a working surface, and a supporting surface, the supporting surface configured to rest upon a stable surface; a compartment formed in the working surface of the body, the compartment configured to hold a syringe therein, the syringe having a distal end with a fluid passage therein and a proximal end with a plunger extending therefrom; a distal protrusion at the distal end of the body adjacent to the compartment, the distal protrusion configured to hold the distal end of the syringe therein; the proximal end of the body further including a cavity therein configured to pass an extender rod therethrough such that the extender rod selectively engages the plunger of the syringe; and a retention lip on the supporting surface of the proximal end of the body, the retention lip configured to prevent the supporting surface of the body from sliding upon the stable surface.

A second aspect of the disclosure provides a syringe dosing system, including: a dosage caddy body containing a cavity which houses a syringe having a plunger to selectively discharge a compound held therewithin, a syringe shield holding the syringe, and an adapter configured to improve the fit of the syringe shield in the cavity; and an extender rod of a predetermined fixed length, the extender rod configured to engage the syringe plunger through pressure, a non-slip adhesive, or locking mechanism.

A third aspect of the disclosure provides a device, including: a caddy body; a body having a proximal end, a distal end, a working surface, and a supporting surface, the supporting surface configured to rest upon a stable surface; a compartment formed in the working surface of the body, the compartment configured to hold a syringe therein, the syringe having a distal end with a fluid passage therein and a proximal end with a plunger extending therefrom; a syringe retaining means at the distal end of the body adjacent to the compartment, the syringe retaining means for holding the distal end of the syringe therein; a passage means in the proximal end of the body, the passage means for passing an extender rod therethrough such that the extender rod selectively engages the plunger of the syringe; and a retention means on the supporting surface of the body, the retention means for preventing the supporting surface of the body from sliding upon the stable surface.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 is a perspective view of a dosage caddy, according to embodiments of the disclosure.

FIG. 2 is a front view of the dosage caddy of FIG. 1, corresponding to the distal end of the dosage caddy, according to embodiments of the disclosure.

FIG. 3 is a rear view of the dosage caddy of FIG. 1, corresponding to the proximal end of the dosage caddy, according to embodiments of the disclosure.

FIG. 4 is a top view of the dosage caddy of FIG. 1, corresponding to the working surface of the dosage caddy, according to embodiments of the disclosure.

FIG. 5 is a front cross-sectional view of the dosage caddy taken along line 5-5 in FIG. 4, according to embodiments of the disclosure.

FIG. 6 is a side view of the dosage caddy of FIG. 1, according to embodiments of the disclosure.

FIG. 7 is a side view of a dosage caddy positioned on a surface and including a syringe and a plunge rod, according to embodiments of the disclosure.

FIG. 8 is a top view of the dosage caddy, the syringe, and the plunge rod of FIG. 7, according to embodiments of the disclosure.

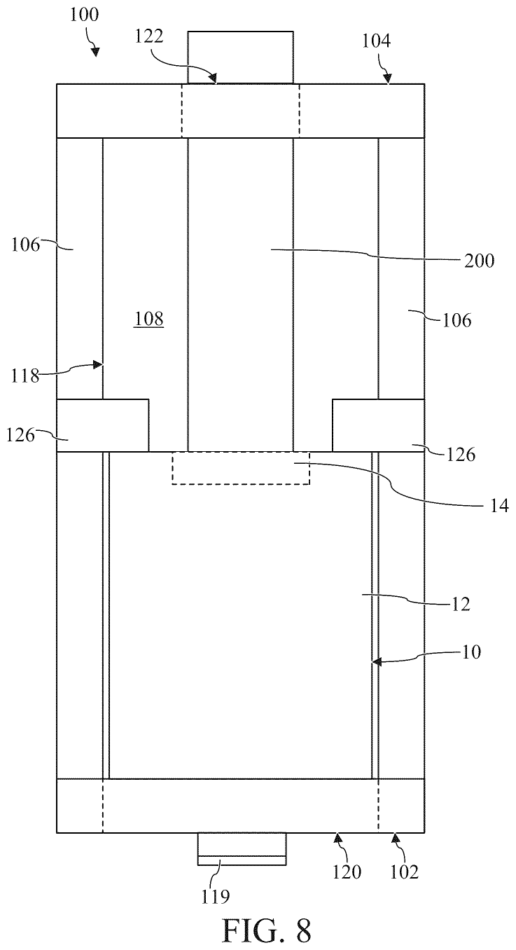

FIG. 9 is a side view of a dosage caddy including a slippage-resistant mechanism, according to additional embodiments of the disclosure.

FIG. 10 is a side view of a dosage caddy including another slippage-resistant mechanism, according to further embodiments of the disclosure.

FIG. 11 is a side view of a dosage caddy including a cover, according to another embodiment of the disclosure.

It is noted that the drawings of the disclosure are not to scale. The drawings are intended to depict only typical aspects of the disclosure, and therefore should not be considered as limiting the scope of the disclosure. In the drawings, like numbering represents like elements between the drawings.

### DETAILED DESCRIPTION

The embodiment of the present disclosure provides a theranostic dose administration caddy to safely and reliably administer commercially available radioligand therapies to patients, whilst reducing radiation exposure of the healthcare staff providing the treatment.

The dose administration caddy discussed herein provides physical support to the standardized radioligand therapy syringes. With the commercial production of standardized radioligand therapies, there is concern about staff radiation exposure to doses administered by intravenous injection from a 20 cc syringe despite using a standard syringe shield and lead-lined gloves. The caddy discussed herein includes a syringe plunger extension that allows healthcare staff to safely and reliably administer the radioligand dose from a stable surface and appropriate distance. Additionally, the dose administration caddy discussed herein allows for a highly adaptable set up with ease of use, simplicity, and a rugged design which reduces radiation dose to staff.

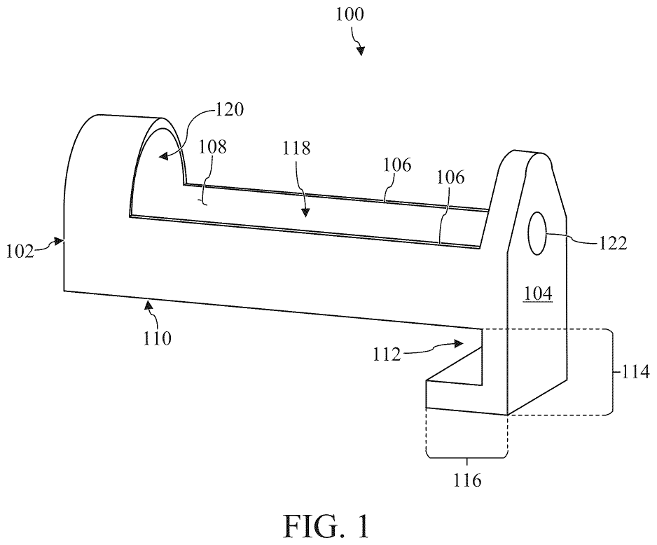

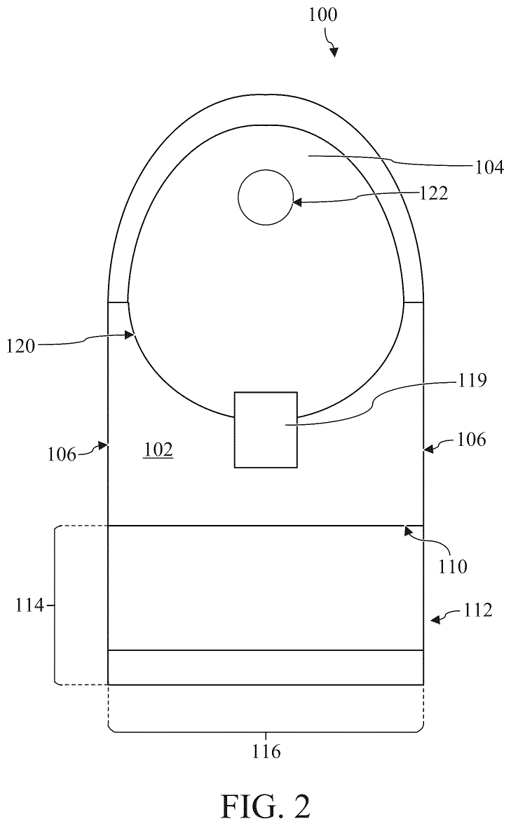

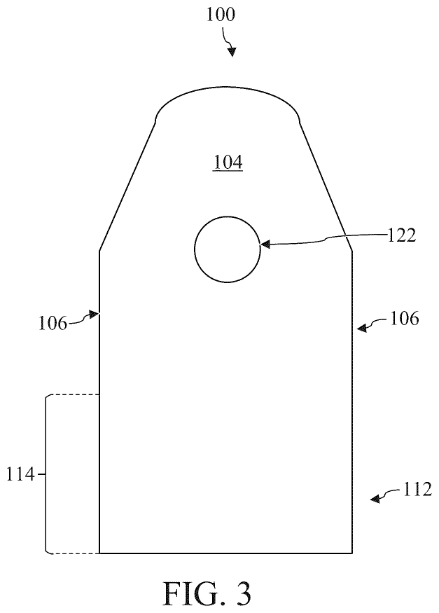

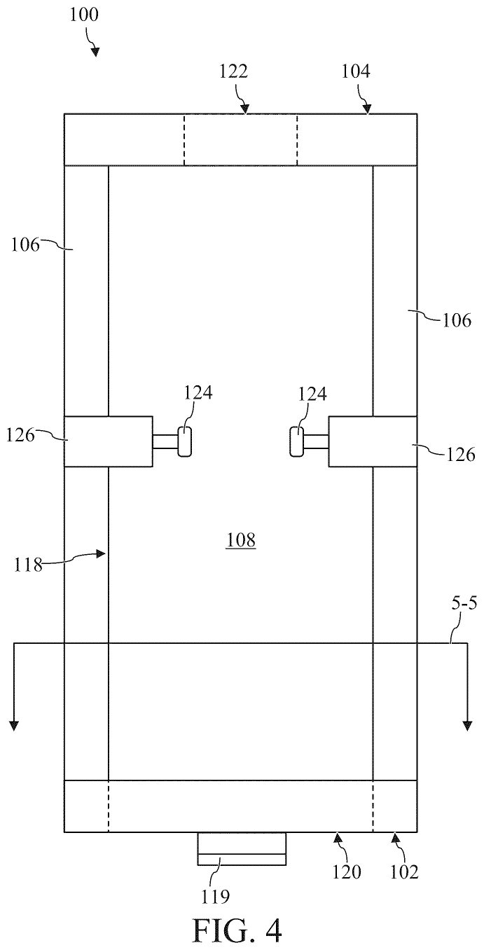

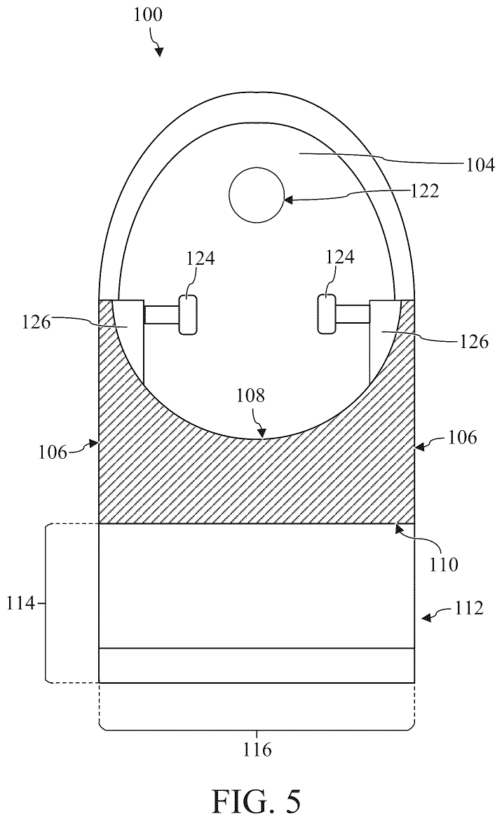

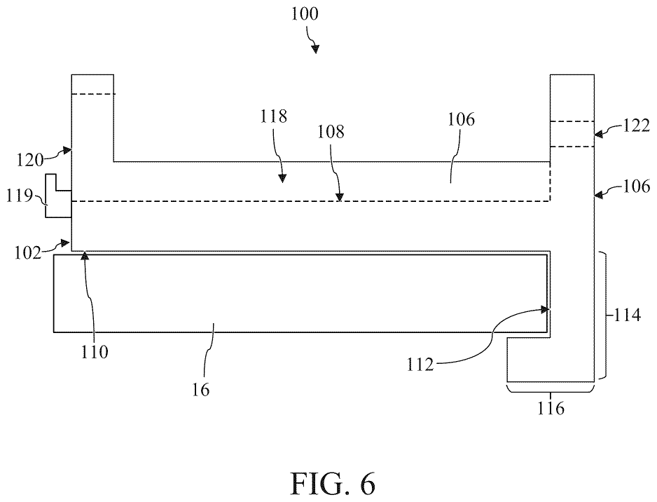

FIGS. 1-6 show various views of a dosage caddy 100 according to embodiments of the disclosure. More specifically, FIG. 1 shows a perspective view of a dosage caddy 100, FIG. 2 shows a front or distal view of dosage caddy 100, FIG. 3 shows a rear or proximal view of dosage caddy 100, FIG. 4 shows a top view of dosage caddy 100, FIG. 5 shows a cross-sectional front view of dosage caddy 100 taken along line CS1-CS1 in FIG. 4, and FIG. 6 shows a side view of dosage caddy 100. In exemplary embodiments, dosage caddy 100 includes a distal end 102 and a proximal end 104 formed, positioned, and/or disposed opposite distal end 102. The proximal end 104 and distal end 102 each contain between them connecting portions or side walls 106 (hereafter, “side walls 106”). That is, in exemplary embodiments two side walls 106 each extend between distal end 102 and proximal end 104 of dosage caddy 100, adjacent to one another.

In the exemplary embodiment shown in FIG. 1, dosage caddy 100 has a working surface 108 and a supporting surface 110 formed, positioned, and/or disposed opposite working surface 108. Additionally, working surface 108 and supporting surface 110 are also formed adjacent to proximal end 104 and distal end 102, respectively. In exemplary embodiments, working surface 108 is substantially rounded and/or curved, or alternatively is substantially flat. Working surface 108 is configured to support a syringe, for dose administration, as discussed herein. Additionally, and as discussed herein, supporting surface 110 is substantially flat to position and/or aid in securing dosage caddy 100 on a stable surface (see, FIG. 6). As such in FIGS. 1 and 5, each of the two side walls 106 extend substantially and/or at least partially between working surface 108 and supporting surface 110.

Dosage caddy 100 includes a retention lip 112 attached to the supporting surface 110 on the proximal end 104 of the caddy 100. Retention lip 112 is formed integral with proximal end 104 of dosage caddy 100 and extends substantially from supporting surface 110, opposite working surface 108. Additionally as shown, at least a portion of retention lip 112 extends substantially perpendicular to support surface 110 of caddy 100. In the exemplary embodiment shown in FIGS. 1, 2, 5, and 6, retention lip 112 includes an extension portion 114 extending perpendicular and/or adjacent to supporting surface 110, and a protrusion portion 116 extending from extension portion 114. Specifically, and as shown, protrusion portion 116 is formed substantially adjacent to and/or separated from supporting surface 110 by extension portion 114. Additionally, protrusion portion 116 of retention lip 112 extends substantially perpendicular to extension portion 114 and/or substantially parallel to supporting surface 110, toward distal end 102. In other exemplary embodiments (not shown), protrusion portion 116 of retention lip 112 extends away from distal end 102 and proximal end 104, respectively, or alternative, can extend toward distal end 102 and away from proximal end 104.

A cavity or hollow compartment 118 (hereafter, “cavity 118”) of caddy 100 is formed between distal end 102 and proximal end 104. More specifically, cavity 118 is defined by and/or formed between, distal end 102, proximal end 104, side walls 106, and working surface 108, respectively. Cavity 118 is also formed opposite supporting surface 110.

In exemplary embodiments, cavity 118 is configured to receive a syringe shield, within which rests a syringe, for dose administration. The working surface 108 is rounded such that the cavity 118 can circumferentially surround at least a portion of a tubular commercially available syringe shield.

As shown in FIGS. 2, 4, and 5, the distal end 102 of the dosage caddy 100 contains a distal opening 120 configured to receive the distal end of a syringe shield for dose administration. Opening 120 adjacent to and/or partially aligned with cavity 118 is sized to prevent syringe from passing through opening 120. A stabilizing plug 119 (see, FIGS. 2 and 4) protruding from the distal end 102 is configured to further stabilize the syringe shield during dose administration. More specifically, the stabilizing plug 119 extends from the bottom of the distal opening 120 perpendicular to the distal end 102. The stabilizing plug 119 extends outward and away from the distal end 102 and proximal end 104, respectively. In other exemplary embodiments, the stabilizing plug 119 can extend inward and away from the distal end 102 and toward the proximal end 104.

The proximal end 102 of the dosage caddy 100 contains a proximal aperture 122 to facilitate dose administration. More specifically, and as shown in FIGS. 1, 2, 3, and 5, proximal aperture 122 extends and/or is formed through proximal end 102 partially aligned with cavity 118, formed adjacent and/or above retention lip 112.

FIG. 4 shows an exemplary embodiment of a dosage caddy 100 shown in FIG. 1. More specifically, FIG. 4 shows a top view of an embodiment of the dosage caddy 100, corresponding to the working surface 108 of the dosage caddy 100. The working surface contains a cut-out through which the cavity 118 can be accessed. The cut-out in the working surface 108 is to facilitate the placement of the syringe shield 12 into the cavity 118. Within the cavity 118, the inner surface of the side walls 106 may include adapters 124 to stabilize and/or hold of the syringe shield 12 within. In an exemplary embodiment, adapters 124 are formed as adjustable, screw-like components to customize the hold and stability of the dosage caddy 100 to variable sizes of the syringe shield 12. In other examples, adapters 124 are formed as static plugs, bars, or any other suitable mechanism that allows the tightening of the grip of the dosage caddy 100 on the syringe shield 12.

FIG. 5 shows a cross-sectional view of an embodiment of a dosage caddy 100 taken from 5-5 in FIG. 4. FIG. 5 shows an alternative view of the dosage caddy 100 where the cross-section is taken through the cavity 118. The cavity 118 is bounded by a working surface 108, opposing side walls 106, and a supporting surface 110. The cavity 118 is dimensioned and/or configured to securely and/or stably receive a tubular syringe shield, wherein the supporting surface 110 is formed with a complementary rounded contour to facilitate the circumferential engagement of a tubular syringe shield. In certain embodiments, the cavity 118 can be further configured to include one or more adapters 124, as discussed herein, protruding perpendicularly from side walls 106. These adapters 124 are configured to accommodate and retain syringe shield 12 of varying diameters, thereby enhancing adaptability and ensuring a snug and stable fit across a range of syringe shield 12 sizes.

The theranostics dose syringe 10 is disposed within a commercially available tubular syringe shield 12. The syringe 10 includes a distal end containing a fluid passage and a proximal end operatively associated with a syringe plunger 14, wherein the distal end of syringe 10 is the aligned with distal end 102 of caddy and the proximal end of syringe 10 aligned with the proximal end 104 end of caddy. A protrusion 126 positioned at the distal end 102 of the caddy, together with the adapters 124 located within the cavity 118, the rounded working surface 108, and the complementary rounded supporting surface 110 are collectively configured to stably hold the syringe shield 12 containing the syringe 10 during dose administration.

FIG. 6 shows a lateral view of an embodiment of a dosage caddy 100 as shown in FIG. 1, wherein the dosage caddy 100 is positioned securely on a stable surface 16 by way of a retention lip 112. As described above, the retention lip 112 extends substantially from the junction of the proximal end 104 and supporting surface 110 of the dosage caddy 100. However, in alternative embodiments, the dosage caddy 100 may be manufactured such that the retention lip 112 is instead attached to the junction of the supporting surface 110 and the distal end 102 of the dosage caddy 100. In such an embodiment (not shown), the protrusion portion 116 of retention lip 112 extends substantially perpendicular to extension portion 114 and/or substantially parallel to supporting surface 110, toward proximal end 104. In other exemplary embodiments (not shown), protrusion portion 116 of retention lip 112 extends away from distal end 102 and proximal end 104, respectively, or alternatively, can extend away from distal end 102 and toward proximal end 104.

Dosage caddy 100 as shown in FIGS. 1-6 is formed as a single, unitary component, where each of the plurality of components and/or features are integrally formed with one another. In other exemplary embodiments, dosage caddy 100 is formed from a plurality of distinct portions and/or components that are affixed, bonded, and/or coupled together to form dosage caddy 100. In non-limiting examples, dosage caddy 100 is produced from commercially available 3D-printing material such as Acrylonitrile Butadiene Styrene (ABS) or Polylactic Acid (PLA). The body of the dosage caddy 100 can be produced to be radiation-resistant by adding tungsten or bismuth oxide to enhance the shielding effect. can further isolate the radiation from the therapy within the dosage caddy during dose administration. Although various examples are given for the 3D-printing materials and radiation-resistant materials, it is understood that the listed materials are exemplary, and any materials suitable for the manufacturing of a radiation-resistant enclosed dosage caddy 100 may be used. Additionally, although discussed herein as being formed using 3D-printing, it is understood that dosage caddy 100 can be formed using any suitable manufacturing techniques and/or processes including, but not limited to, casting, molding, material removal processes (e.g., machining, milling, etc.), or any other suitable manufacturing process.

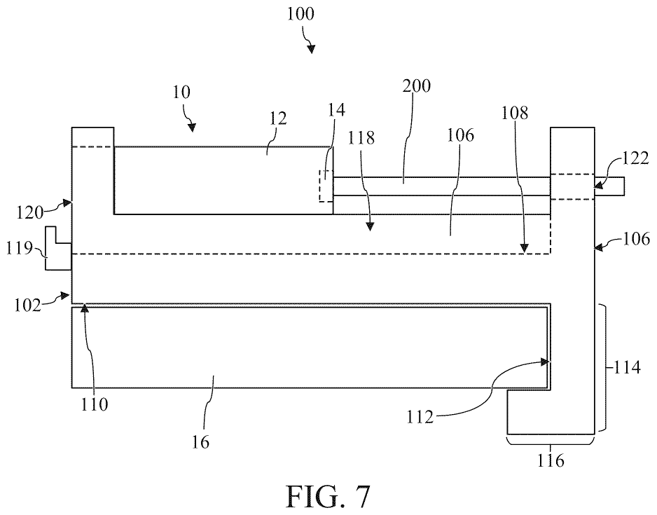

FIG. 7 illustrates a lateral view of an embodiment of a dosage caddy 100 as shown in FIG. 1, wherein the cavity 118 houses a plunger extender rod 200 of predetermined length. The plunger extender rod 200 is movable through an aperture 122 formed in the proximal end 104 of the dosage caddy 100. The plunger extender rod 200 is configured to operably and reliably engage with a syringe plunger 14 through applied pressure, a non-slip adhesive, or a locking mechanism. It is understood that these methods are exemplary embodiments, and any connection mechanism between the plunger extender rod 200 and the syringe plunger 14 that facilitates the safe delivery of the dose from the syringe 10 may be employed. Once the plunger extender rod 200 is engaged with the syringe plunger 14 within the cavity 118 of the dosage caddy 100, the rod 200 may be advanced by healthcare personnel from a safe distance to facilitate the safe and controlled discharge of the compound contained within the syringe 10. Moreover, the distal end 102 of the dosage caddy 100 contains a stabilizing plug 119 configured to prevent the syringe shield 12, and the syringe 10 therein from sliding forward when pressure is applied from the plunger extender rod 200 for dose administration.

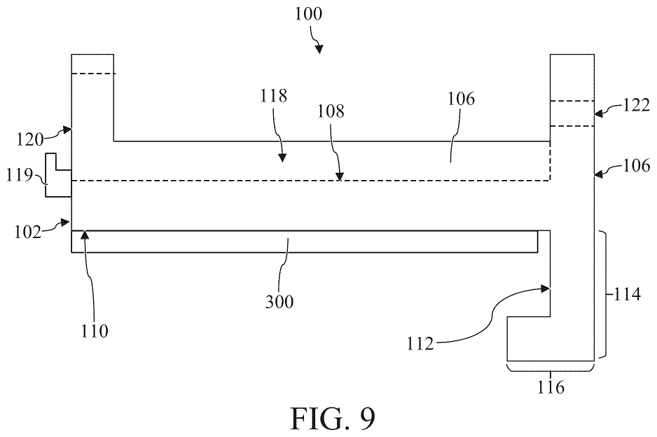

FIG. 9 shows a slippage-resistant mechanism 300, which may comprise, by way of non-limiting example, a non-slip coating, contained on and/or disposed over supporting surface 110 of the dosage caddy 100. The slippage-resistant mechanism 300 is positioned in between the supporting surface 110 of the dosage caddy 100 and the stable surface 16 so as to further ensure the stability of the dosage caddy 100 during dose administration. In exemplary embodiments, the slippage-resistant mechanism 300 may be composed of, but not limited to, silicone rubber, medical-grade pressure-sensitive adhesives, serrated or ribbed polymer surfaces, or any other suitable material to prevent or reduce slippage of caddy 100 during operation.

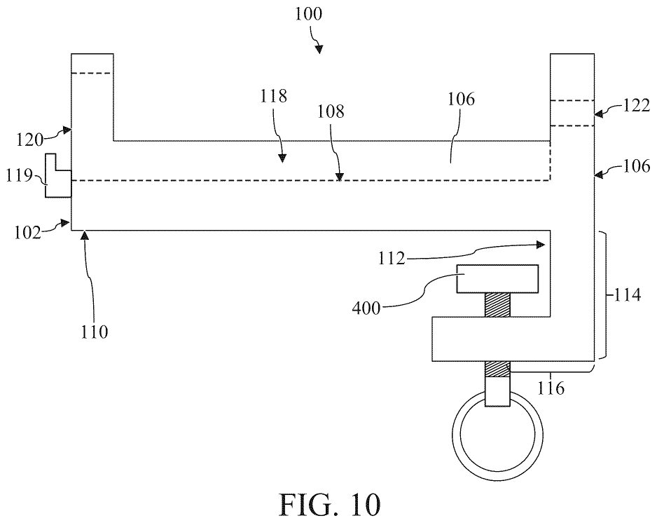

Furthermore, FIG. 10 shows that the protrusion portion of the retention lip 112 is configured to contain a clamping mechanism 400. The clamping mechanism 400 is operable to tighten or secure the engagement of the dosage caddy 100 with the stable surface 16 to achieve a more secure fit, thus increasing the overall stability of the dosage caddy 100 and preventing it from slippage when axial pressure is applied for dose administration. In certain embodiments, the clamping mechanism 400 may comprise a spring-loaded clamp, a screw clamp, a wedge-type locking element, or a friction enhancing compression plate. In other embodiments, the clamping mechanism 400 may include a pivoting or articulating jaw configured to adapt to stable surfaces 16 of varying thickness or textures.

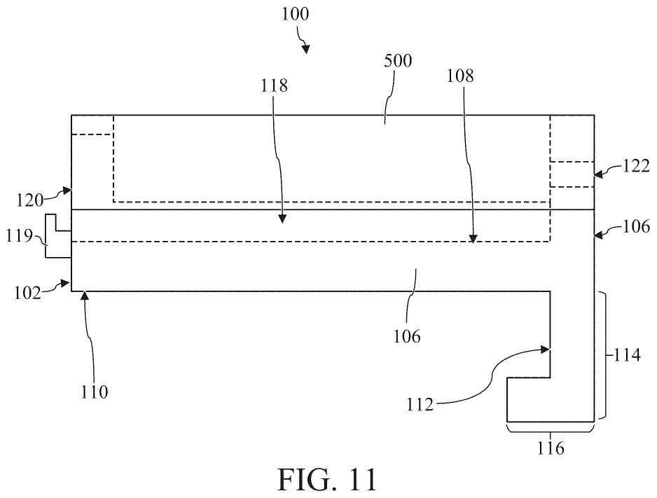

FIG. 11 shows a lateral view of an embodiment of the dosage caddy 100, wherein the cut-out in the working surface 108 and the cavity 118 may be selectively covered by a hinged lid 500. The hinged lid 500 may be composed from radiation-resistant materials and is configured to further isolate or attenuate the radiation emanating from the therapy contained within the dosage caddy 100 during dose administration. In certain embodiments, the hinged lid 500 may include a mechanical hinge, living hinge, spring-biased hinge, or latch mechanisms to ensure secure closure and controlled access to the cavity 118. The hinged lid 500 may additionally be provided with a locking feature, gasket, or sealing interface to enhance containment, reduce radiation leakage, and improve overall structural integrity of the dosage caddy 100 during dose administration.

The foregoing drawings show some of the processing associated according to several embodiments of this disclosure. In this regard, each drawing or block within a flow diagram of the drawings represents a process associated with embodiments of the method described. It should also be noted that in some alternative implementations, the acts noted in the drawings or blocks may occur out of the order noted in the figure or, for example, may in fact be executed substantially concurrently or in the reverse order, depending upon the act involved. Also, one of ordinary skill in the art will recognize that additional blocks that describe the processing may be added.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. “Approximately” and/or “substantially” as applied to a particular value of a range applies to both values, and unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A dosage caddy, comprising:
a body having a proximal end, a distal end, a working surface, and a supporting surface, the supporting surface configured to rest upon a stable surface;
a compartment formed in the working surface of the body, the compartment configured to hold a syringe therein, the syringe having a distal end with a fluid passage therein and a proximal end with a plunger extending therefrom;
a distal protrusion at the distal end of the body adjacent to the compartment, the distal protrusion configured to hold the distal end of the syringe therein;
the proximal end of the body further including a cavity therein configured to pass an extender rod therethrough such that the extender rod selectively engages the plunger of the syringe; and
a retention lip on the supporting surface of the proximal end of the body, the retention lip configured to prevent the supporting surface of the body from sliding upon the stable surface.

2. The dosage caddy of claim 1, wherein the cavity further configured to hold a syringe shield holding a syringe therewithin.

3. The dosage caddy of claim 1, further comprised of one or more of: Polylactic Acid (PLA), Acrylonitrile Butadiene Styrene (ABS).

4. The dosage caddy of claim 1, wherein the body is comprised of radiation-resistant material.

5. The dosage caddy of claim 1 wherein the supporting surface of the body includes a slippage-resistant mechanism.

6. The dosage caddy of claim 5, wherein the slippage-resistant mechanism is one of a material selected from a group consisting of silicone rubber, medical-grade pressure-sensitive adhesives, and serrated or ribbed polymer surfaces.

7. The dosage caddy of claim 1, wherein the stable surface has an edge thereof, and the retention lip extends perpendicularly from the supporting surface such that the dosage caddy securely attaches to the edge of the stable surface.

8. The dosage caddy of claim 1, further comprising an extender rod.

9. The dosage caddy of claim 8, wherein the syringe further containing a radioactive compound, and the extender rod further having a predetermined length thereof based upon the radioactive compound in the syringe.

10. the dosage caddy of claim 8, wherein the extender rod further configured to engage with the plunger of the syringe.

11. The dosage caddy of claim 10, wherein the extender rod engages the plunger of the syringe with pressure, a non-slip adhesive, or locking mechanism.

12. A syringe dosing system, comprising:
a dosage caddy body containing a cavity which houses a syringe having a plunger to selectively discharge a compound held therewithin, a syringe shield holding the syringe, and an adapter configured to improve a fit of the syringe shield in the cavity; and
an extender rod of a predetermined fixed length, the extender rod configured to engage the plunger through pressure, a non-slip adhesive, or locking mechanism.

13. The syringe dosing system of claim 12, wherein the dosage caddy body can be securely fastened to a planar surface.

14. The syringe dosing system of claim 12, wherein an inner retention lip formed on a distal end of the dosage caddy body prevents the syringe and syringe shield from sliding forward as the extender rod applies pressure the plunger of the syringe.

15. The syringe dosing system of claim 12, wherein:
the extender rod is moveable forward through an opening in a proximal end of the dosage caddy body to engage the syringe plunger, the extender rod housed in the cavity of the dosage caddy body; and
movement of the extender rod expels contents of the syringe.

16. A device, comprising:
a caddy body;
a body having a proximal end, a distal end, a working surface, and a supporting surface, the supporting surface configured to rest upon a stable surface;
a compartment formed in the working surface of the body, the compartment configured to hold a syringe therein, the syringe having a distal end with a fluid passage therein and a proximal end with a plunger extending therefrom;
a syringe retaining means at the distal end of the body adjacent to the compartment, the syringe retaining means for holding the distal end of the syringe therein;
a passage means in the proximal end of the body, the passage means for passing an extender rod therethrough such that the extender rod selectively engages the plunger of the syringe; and
a retention means on the supporting surface of the body, the retention means for preventing the supporting surface of the body from sliding upon the stable surface.

17. The device of claim 16, wherein the caddy body is formed from additive manufacturing.

18. The device of claim 16, wherein the caddy body further comprises a cavity which houses a syringe, syringe shield and adaptor.

19. The device of claim 16, wherein the retention means is a securing device on the supporting surface of the body.

20. The device of claim 16, wherein the retention means is a high-friction material on the supporting surface of the body.
